(19) United States
(12) Patent Application Publication
(10) Pub. No.: US 2024/0264087 A1
(43) Pub. Date: Aug. 8, 2024

(54) DEVICE FOR MEASURING AT LEAST ONE GASEOUS OR SOLID MATERIAL

(71) Applicant: AVL List GmbH, Graz (AT)

(72) Inventors: Andreas Pein, Paldau (AT); Gerald Steiner, Graz (AT); Mario Schaller, Graz (AT)

(21) Appl. No.: 18/572,928

(22) PCT Filed: Jun. 24, 2022
(86) PCT No.: PCT/AT2022/060214

§ 371 Date: Dec. 21, 2023

(30) Foreign Application Priority Data
Jun. 25, 2021 (AT) A50527/2021

(51) Int. Cl. G01N 21/85; G01N 15/075; G01N 21/27; G01N 21/59; G08B 21/12

(52) U.S. Cl. CPC G01N 21/85; G01N 15/075; G01N 21/27; G01N 21/59; G01N 2021/8578; G01N 2201/0668; G08B 21/12

(57) ABSTRACT

The invention relates to a device for measuring at least one gaseous or solid material in at least one measurement volume at a stationary measurement station, wherein: a light source and at least one detector are provided, and at least one main primary beam can be emitted from the light source to at least one beam splitter unit; the at least one beam splitter unit is disposed at a first distance from a first reflection region of a reflection unit, and the beam splitter unit splits the main primary beam into at least one first partial beam oriented through the measurement volume toward the first reflection region and at least one secondary primary beam oriented in a different direction than the main primary beam; at least one deflecting unit is disposed at a second distance from a second reflection region, and the at least one secondary primary beam can be directed to the deflecting unit by means of the beam splitter unit and the at least one secondary primary beam can be directed, as a second partial beam, through the at least one measurement volume toward the second reflection region by means of the deflecting unit; and each of the at least one measurement volume is disposed between the beam splitter unit and/or the deflecting unit and the associated reflection regions and is at least partly delimited by the beam splitter unit and/or the deflecting unit and the associated reflection regions. According to the invention, the first reflection region directs the first partial beam, as a first return beam, through the at least one measurement volume to the at least one detector, the second reflection region directs the second partial beam, as a second return beam, through the at least one measurement volume to the at least one detector, and the at least one detector measures a light property of each return beam, said light property characterizing the at least one gaseous or solid material.

The present invention relates to a device for measuring at least one gaseous or solid material in at least one measurement volume at a stationary measurement station, wherein a light source and at least one detector are provided, and at least one main primary beam can be emitted from the light source to at least one beam splitter unit, wherein the at least one beam splitter unit is arranged at a first distance from a first reflection region of a reflection unit, and the beam splitter unit splits the main primary beam into at least one first partial beam oriented through the measurement volume toward the first reflection region and at least one secondary primary beam oriented in a different direction than the main primary beam, wherein at least one deflecting unit is arranged at a second distance from a second reflection region, and the at least one secondary primary beam can be directed to the deflecting unit by means of the beam splitter unit, and the at least one secondary primary beam can be directed, as a second partial beam, through the at least one measurement volume toward the second reflection region by means of the deflecting unit, and wherein the at least one measurement volume is disposed in each case between the beam splitter unit and/or the deflecting unit and the associated reflection regions, and is at least partly delimited by the beam splitter unit and/or the deflecting unit and the associated reflection regions.

The invention also relates to a method for measuring at least one gaseous or solid material in at least one measurement volume which is formed between a beam splitter unit and a first reflection region and/or a deflection unit and a second reflection region.

Emissions of materials in exhaust gases, especially in private transport, are a much-discussed topic due to the increasing number of vehicles, not only in light of global warming, but also in light of the health burden on humans from nitrogen oxides, partially combusted fuel components, and fine dust particles. Developments in recent decades aimed on the one hand to avoid the emission of partially combusted compounds by the mandatory installation of catalytic converters in gasoline engines, and on the other hand to avoid the emission of nitrogen oxides by catalytic converters in diesel engines. The permissible values of emitted materials are often determined by national and supranational standards.

Nevertheless, there are still vehicles in use in public spaces today which, although they met the legal standards for exhaust gas reduction at the time of registration, are considered to be high emitters when used over a longer period of time. The reason for this can be, for example, the failure to retrofit a catalytic converter or a lack of maintenance, for example if a diesel catalytic converter is not topped up with urea, and an SCR (selective catalytic reduction) catalytic converter is no longer functioning properly. Among other things, this can also be due to lack of knowledge regarding the (non-) functionality of these components while driving.

Exhaust gas measurements are largely limited to systems which measure materials in the exhaust gas, such as gaseous materials or particles, in the vehicle itself, for example in or after the exhaust pipe. However, these systems are limited to a small number of test vehicles and can therefore not give a representative picture of a plurality of different vehicles in real operation. Exhaust gas measurements within the framework of regular checking of the vehicle in a repair shop are also not representative because such checks are carried out only at long intervals. For this reason, the attempt is being made to enable exhaust gas measurements of vehicles in real operation in public spaces. This so-called “remote sensing,” also in the sense of “real driving emissions” (RDE) measurements, is carried out at a stationary measuring station and can, for example, be attached to advantageously pre-installed infrastructure such as toll booths, street lamps, bridges or even building facades in the city and the like. This could be used, for example, to notify owners of vehicles with high emissions and/or to provide obligatory maintenance. However, care must be taken when setting up the devices to ensure a suitable measurement station in order to obtain representative results. In general, intersections with traffic lights and accordingly a potential standstill of vehicles should be avoided. Furthermore, it has been shown that a slight slope of the road at the measurement station is suitable for generating a positive motor load.

In remote sensing, a light source is often used which emits a characteristic wavelength or wavelength range(s) in order to detect a gaseous material such as carbon monoxide or nitrogen oxides. A detector enables, for example, a measurement of the attenuation of the light that is transmitted through the plume. However, it can also be provided to measure particles, such as soot particles, as a material. This can then be realized, for example, via light scattering or by measuring the attenuation of the return beam in relation to the radiated light.

Such remote sensing applications are known from WO 2010/026579 A2, CN 206756689 U, US 2003/089854 A1, U.S. Pat. No. 5,401,967 A or CN 208060384 U.

However, the reliable measurement of such materials in exhaust plumes can lead to various difficulties. On the one hand, the emissions of materials from different engines or also other energy systems, such as fuel cells, are quite different and must be measurable with the same system. Furthermore, the concentration range to be measured fluctuates very strongly and is dependent on the vehicle class to be measured (e.g., truck vs motorcycle). Especially low concentrations cause problems during evaluation. Differences in the operating temperature of an engine can also result in differences in the materials to be measured.

A number of the above-mentioned cross-influences and inaccuracies could be eliminated by multiple measurements of materials in an exhaust plume. In this case, it may be possible to measure an exhaust plume several times in succession, or also to measure an exhaust plume at multiple points.

EP 3 702 757 A1 discloses a multiple measurement system for remote sensing in public spaces, whereby exhaust plumes are measured on the road and above the road via beam splitter units and positioning units, and wherein the emitted light is captured via collectors on the road surface and directed to a central detector. This is disadvantageous for exhaust gas components in low concentration because the emitted light passes through the exhaust plume only once at every point. Since the collectors are arranged on a road surface, contamination inevitably leads to a reduction in the detected signal. This generates different changes in the measurement and makes it necessary to regularly clean the measuring device.

The object of the present invention is therefore to provide a low-maintenance remote sensing device for multiple measurement of a gaseous or solid material in a measurement volume which is capable of also measuring materials at a low concentration.

According to the invention, this object is achieved by a device mentioned at the beginning in that the first reflection region is provided in order to direct the first partial beam as a first return beam through the at least one measurement volume to the at least one detector, in that the second reflection region is provided in order to direct the second partial beam as a second return beam through the at least one measurement volume to the at least one detector, and in that the at least one detector is provided to measure a light property of each return beam characterizing the at least one gaseous or solid material.

The device according to the invention is advantageous because the emitted partial beams and return beams pass through the measurement volume at least twice and accordingly, due to an integral measurement, a more accurate measurement of a gaseous or solid material is enabled in the measurement volume. Likewise, materials in a lower concentration in the measurement volume can accordingly be measured. Measuring can be at at least two points in the same measurement volume or in at least two separate measurement volumes, which also makes the device flexible to use. A reflection unit, which is usually arranged on a surface to be measured, and therefore is directly exposed to external influences such as vehicle traffic, can be exchanged easily and cheaply, whereby possible maintenance times and associated failures due to damaged reflection units can be kept low.

Depending on the application and requirements, the respective return beam is advantageously directed directly to a detector via the reflection regions or is deflected back to the at least one detector via the beam splitter unit, or via the deflection unit, or via the deflection unit and the beam splitter unit. This can also be made dependent on where the at least one detector can be arranged or may already be arranged.

In one variant of the invention, the detector is arranged in the deflection unit or the beam splitter unit, also depending on the application and the requirements, or also a detector each in both units. An arrangement of the detector together with the light source in a housing is also possible. Through these various possibilities, the device can be adapted very flexibly to the particular application, which facilitates use.

In order to easily increase the number of measurement points for measuring at least one gaseous or solid material in at least one measurement volume, it can be provided to arrange a second beam splitter unit at a third distance from a third reflection region. This second beam splitter unit is arranged between the first beam splitter unit and the deflection unit and receives the secondary primary beam from the first beam splitter unit and divides it into a third partial beam oriented in the direction of the third reflection region and a second secondary primary beam oriented in the direction of the deflection unit. In other words, the second beam splitter unit is arranged between the first beam splitter unit and the deflection unit when viewed in the direction of radiation of the secondary primary beam. Another measurement point is consequently realized by the third partial beam.

An increase in the number of measurement points can also be achieved in that a beam splitter unit divides the obtained main primary beam or secondary primary beam into a first or third partial beam and into multiple secondary primary beams oriented in different directions, wherein the beam splitter unit directs each secondary primary beam to another beam splitter unit or a deflection unit. In this case, the directions of the secondary primary beams are different from one another, but preferably also different from the direction of the first and/or third partial beam.

In order to keep the number of beam splitter units low, one variant of the invention provides that at least one beam splitter unit is arranged so as to be pivotable about an axis in order to selectively guide the generated secondary primary beam to a plurality of deflection units and/or additional beam splitter units, wherein the axis is designed to extend normally to a reflection region or to a surface on which the reflection unit is arranged. A secondary primary beam can accordingly be easily guided with a beam splitter unit to multiple additional beam splitter units and/or deflection units.

By means of a positioning optical unit in a beam splitter unit and/or deflection unit, in another variant of the invention, the direction of a generated partial beam is adjustable, in particular deflectable at an angle deviating from a normal to the respective reflection region. In other words, in this variant, a positioning optical unit is provided in at least one beam splitter unit and/or deflection unit, and the positioning optical unit directs the first partial beam oriented in the direction of the respective reflection region in an angle deviating from a normal to the respective reflection region.

However, the positioning optical unit can also be used to increase the number of passages of a partial beam through the measurement volume and accordingly to increase the sensitivity of the measurement. The multiple passage through the measurement volume results in an integral measurement because the light beam is repeatedly influenced by the at least one gaseous or solid material in the measurement volume. This advantage can be realized by arranging an opposite second reflection unit with an opposite twin reflection region facing the respective reflection region in at least one beam splitter unit and/or deflection unit at a distance from the respective reflection region, wherein the respective reflection region reflects the partial beam to the opposite twin reflection region, and the opposite twin reflection region reflects the partial beam back to the respective reflection region, wherein the at least one detector is provided to detect the partial beam after a plurality of such reflections as a return beam, or a deflection mirror is provided which deflects the partial beam after a plurality of such reflections as a return beam, and the at least one detector detects the return beam after a number of reflections between the reflection unit and the opposite reflection unit.

In order to be able to easily adapt the number of passages, the angle of the partial beam is preferably adjustable by the positioning optical unit.

If at least one partial beam, and/or a return beam, and/or the main primary beam, and/or a secondary primary beam is divided into individual light packets by a modulation unit, the detection of the return beams in the detector can be simplified. Preferably, at least one modulation unit is therefore provided in the device in order to divide at least one partial beam, and/or a return beam, and/or the main primary beam, and/or a secondary primary beam into individual light packets. If the light packets are additionally modulated offset in time, the time-offset light packets can be detected by the detector. Preferably, at least two light packets are therefore offset in time. Hence, for example, a detector can easily measure in a spatially resolved manner if it always receives only one light packet at a time.

In order to realize multiple measurement stations with a beam splitter unit or deflection unit, in one variant of the invention, a multiplexer unit is provided in a beam splitter unit and/or deflection unit and divides the obtained main primary beam or secondary primary beam into a plurality of partial beams, and the multiplexer unit directs the plurality of partial beams to different locations of the respective reflection region which reflects the plurality of partial beams as a plurality of return beams and transmits the plurality of return beams to the multiplexer unit, and the multiplexer unit transmits the plurality of reflected return beams to at least one detector. In this way, a spatially resolved measurement of the measurement volume can be achieved. In this case, it can be provided that a dedicated detector is provided for at least two of the plurality of return beams, whereby the return beams can be detected at the same time.

In another variant of the invention, at least two of the plurality of partial beams in the multiplexer unit are each guided via a dedicated optical path, wherein the optical paths have different optical path lengths, and a common detector is provided for the at least two resulting return beams. Due to the different optical path lengths, the return beams arrive at different points in time in the detector, which enables a simple separation of the detection of the individual return beams in the detector. Of course, this can also be combined with a modulation of the light beams into individual light packets.

In a particularly advantageous embodiment, at least one imaging unit is provided in the device in order to record at least a part of the exhaust plume from different directions when an exhaust gas cloud is present in the measurement volume, wherein an evaluation unit is provided in order to reconstruct the images recorded by the imaging unit into an image of the at least one part of the exhaust plume, and to determine a passage length of the partial beam and/or of the return beam through the exhaust plume in the measurement volume from the image of the at least one part of the exhaust plume. The at least one detector which detects the return beam, determines a decrease in intensity of the detected return beam due to the at least one gaseous or solid material. Then from the decrease in intensity and the determined passage length, the evaluation unit determines a concentration of the at least one gaseous or solid material in the measurement volume. This allows a more accurate determination of a concentration of a gaseous or solid material in the measurement volume. A plurality of cameras and/or one or more lidar units can advantageously be used as an imaging unit. The plurality of cameras and/or the one or more lidar units are preferably arranged at different positions. In other words, the plurality of cameras and/or the one or more lidar units are arranged in such a way that the part of the exhaust plume can be imaged from different spatial directions. In this way, they can image the part of the exhaust plume from different directions.

In order to protect sensitive reflection regions from contamination or damage, a protective film is advantageously arranged replaceably over reflection regions. In this way, necessary maintenance intervals can be reduced.

The object described above is also achieved according to the invention by a method mentioned at the outset in that:
a main primary beam is emitted from a light source, and the main primary beam is guided to the at least one beam splitter unit in which the main primary beam is divided into at least one first partial beam oriented in the direction of the first reflection region and through the at least one measurement volume and a secondary primary beam, wherein
the secondary primary beam is deflected in the deflection unit into a second partial beam oriented in the direction of the second reflection region and through the at least one measurement volume, wherein
the first partial beam is reflected at the first reflection region and is deflected back as a first return beam through the at least one measurement volume to the at least one detector, wherein
the second partial beam is reflected at the second reflection region and is deflected back as a second return beam through the at least one measurement volume to the at least one detector, and
a light property of each return beam that characterizes the at least one gaseous or solid material is measured with the at least one detector.

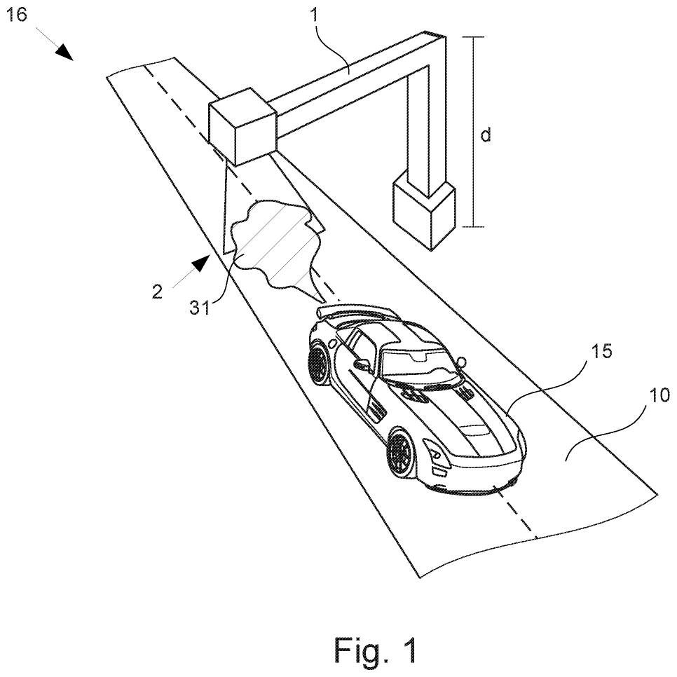

The present invention is described in greater detail below with reference to FIGS. 1 through 9, which show schematic and non-limiting advantageous embodiments of the invention by way of example. In the figures:

FIG. 1 shows the principle of remote sensing according to the prior art,

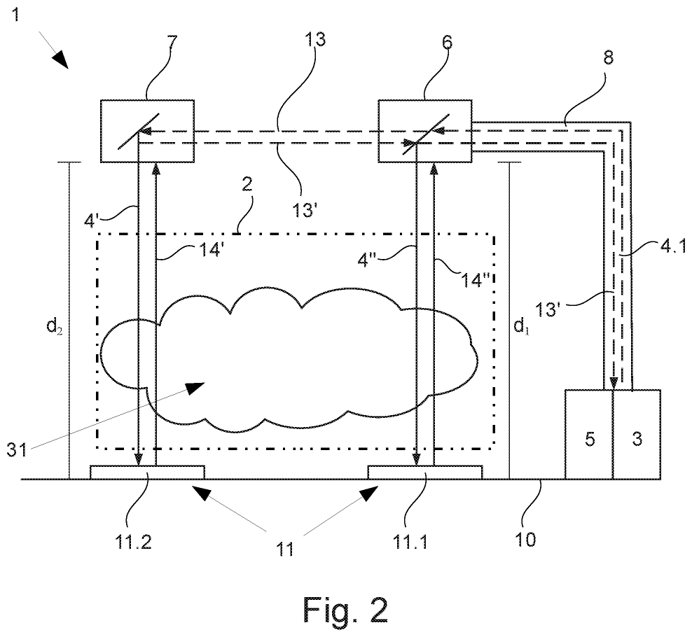

FIG. 2 shows an embodiment according to the invention of a device for measuring at least one gaseous or solid material in a measurement volume,

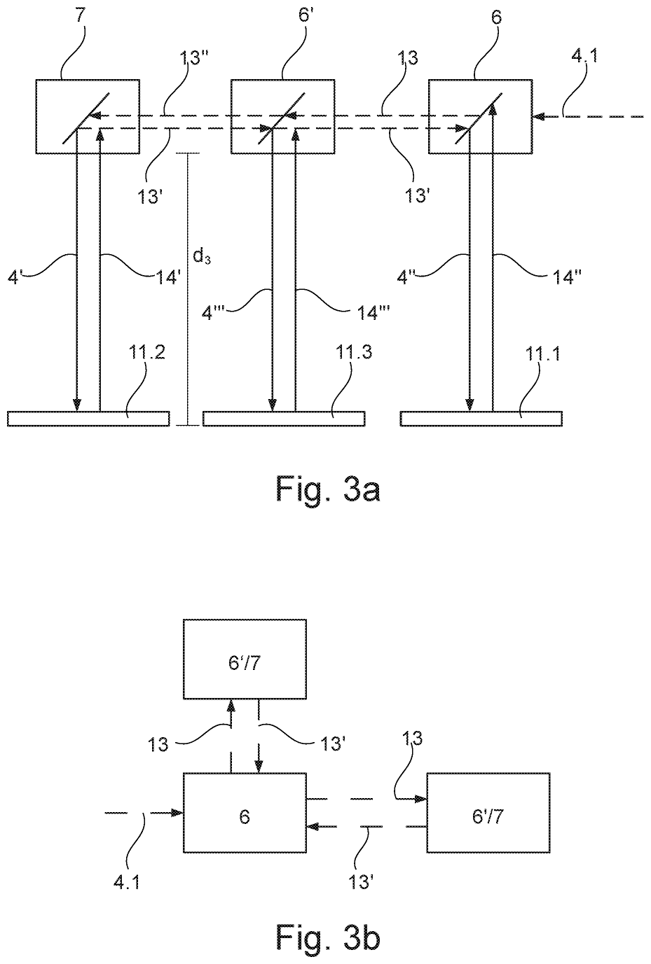

FIGS. 3a and 3b show advantageous embodiments of the device according to the invention,

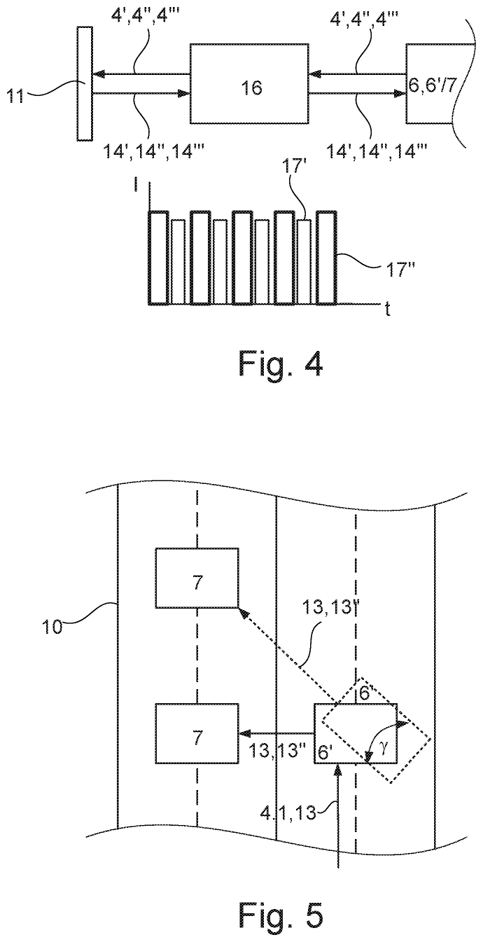

FIG. 4 shows an embodiment with a modulation of the partial beams,

FIG. 5 shows an embodiment with an at least partially pivotable embodiment of the device according to the invention,

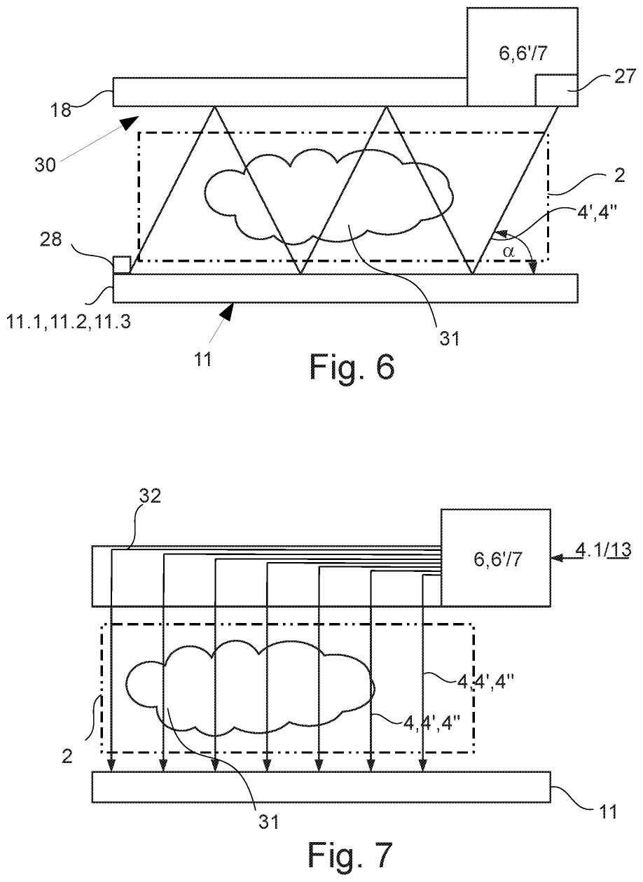

FIG. 6 shows another advantageous embodiment of the measuring unit with a double-mirror system,

FIG. 7 shows another advantageous embodiment of the measurement unit with multiplexer,

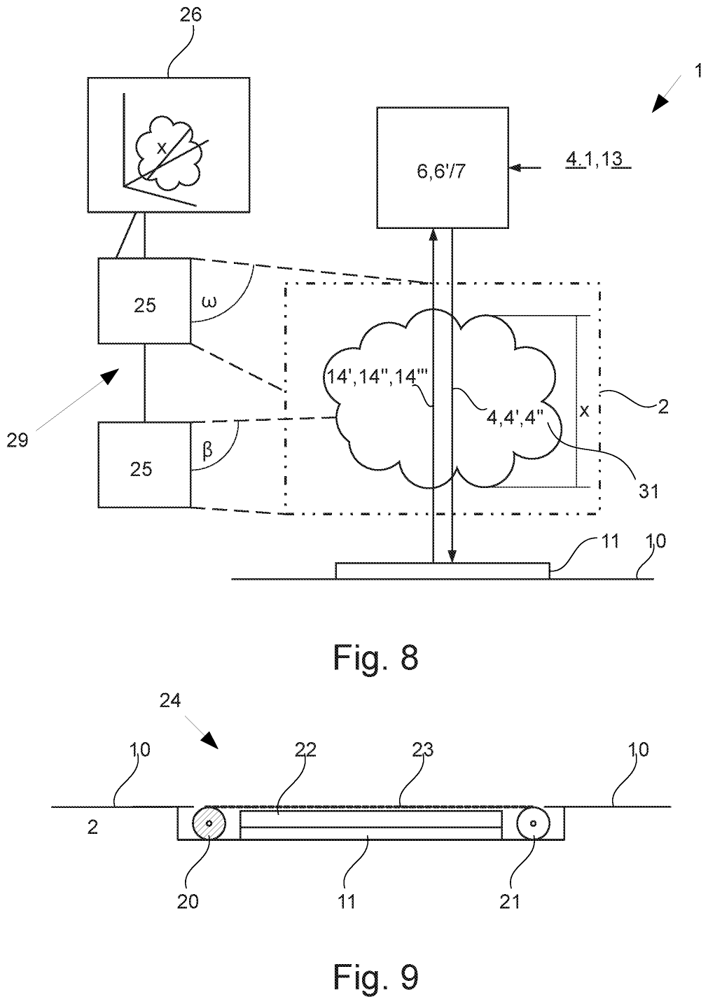

FIG. 8 shows another advantageous embodiment of the measuring unit with concentration measurement, and

FIG. 9 shows protection for the reflection unit.

FIG. 1 shows a device 1 according to the prior art for measuring a gaseous or solid material in a measurement volume 2. An exhaust plume 31 can be located in the measurement volume 2, for example which is emitted in the public space 16 by a vehicle or another emission source 15. In the measurement volume 2, a wide variety of gaseous and solid (e.g., particles) components can arise. For example, an exhaust plume 31 of an automobile can be present in the measurement volume 2. The materials in the measurement volume 2 can originate from any type of emission source 15, for example on a surface 10. In the illustrated FIG. 1, the emission source 15 is a vehicle such as a passenger car, truck, but also a single-track vehicle such as a motorcycle, motorized bicycle, and the like, which have an internal combustion engine. Emissions from another emission source 15, such as fuel cells which usually emit only water vapor and no pollutants, can also be measured via such a device 1. The detection of such emission sources 15 can be helpful in determining, for example, the share of vehicles having low or high emission values in road traffic. The measurement can take place, for example, on a surface 10, for example a road, advantageously at a certain distance d above a surface 10. However, it is also conceivable that the device 1 is arranged on the side of a measurement volume 2, and the measurement takes place parallel to the surface 10, or the device 1 can also be installed in the surface 10 itself. Combinations of measurements from a number of sides are also conceivable.

In addition to use in the automobile industry, other uses in which exhaust plumes 31 with gaseous or solid materials arise as emissions are also conceivable, for example in the process industry. In this case, for example, emissions can be measured in chimneys which can have diameters of a few meters. The measurement volume 7 would thereby be formed in the chimney.

The device 1 can measure a measurement volume 2, for example, also at other locations away from a surface 10. It is conceivable that a device 1 in a measurement volume 2 measures an exhaust plume of an aircraft when starting or landing on a landing strip on an airport. It is also conceivable for an exhaust plume from a ship to be measured, for example in a harbor basin or in a lock.

The invention is not limited to the above-mentioned applications, but rather all possible uses that are apparent to a person skilled in the art are conceivable.

The exhaust plume 31 also does not necessarily have to originate from a vehicle, but can in principle originate from any emission source 15. One example is an exhaust plume 31 from an industrial process which is emitted, for example, from a chimney.

The materials to be measured in the measurement volume 2 can be gaseous materials such as carbon dioxide (CO₂), carbon monoxide (CO), nitrogen oxides (NOx), sulfur dioxide (SO₂), gaseous polycyclic aromatic hydrocarbons (PAH) and the like. However, it is also conceivable to measure solid materials such as solid particles like soot particles in the measurement volume 2. The materials and their concentrations in the measurement volume 2 are usually dependent on the emission source 15, for example the fuel type, the internal combustion engine, the operating state of the internal combustion engine, and the status of a catalytic converter or exhaust gas aftertreatment system (if present). For example, an internal combustion engine which is not yet at operating temperature often emits a higher concentration of partially combusted materials such as polycyclic aromatic hydrocarbons than at normal operating temperature. Likewise, different materials are emitted in different operating states (e.g., given by current rotational speed and current torque).

These gaseous or solid materials in the measurement volume 2 are to be measured according to the invention. “Measuring a material” can mean the detection of the presence of the material in the measurement volume 2, but also the measurement of an amount or concentration of the material in the measurement volume 2.

Remote sensing is based on the fact that a light beam is transmitted through the measurement volume 2, and a light property of the light beam, for example the light intensity, or a wavelength of the light, is changed due to gaseous and/or solid materials in the measurement volume 2. This change can be measured using a detector 5 in order to obtain conclusions about the gaseous or solid materials in the measurement volume 2.

FIG. 2 shows an embodiment of remote sensing according to the invention, with a device 1 for multiple measurement of a solid or gaseous material in at least one measurement volume 2.

For this purpose, a light source 3 is provided in the device 1 and is arranged in spatial proximity to a detector 5 in the shown embodiment. The light source 3 and the detector 5 are preferably arranged in a common housing. The light source 3 can emit monochromatic light, for example, as laser light, which emits a defined wavelength with a predefined light intensity. In particular, quanta cascade lasers (QCL) can be used, but other types and combinations of lasers are also conceivable in order to cover different wavelength ranges. It is also conceivable for the light source 3 to have a polychromatic emitting lamp, such as a lamp in the ultraviolet (UV) or also in the infrared range (IR). A monochromator in the light source 3 or at another suitable location of the device 1 is also conceivable in order to select wavelengths in a targeted manner. A monochromator can be, for example, a Bragg grating, a prism, a movable mirror or an optical filter.

The light source 3 generates a main primary beam 4.1 with a predetermined light intensity and at least one wavelength, and this is guided to a beamsplitter unit 6. For this purpose, the light source 3 can be arranged either in the beamsplitter unit 6 or in the spatial vicinity thereof. It is also possible to guide the main primary beam 4.1 to the beamsplitter unit 6 via a light transport unit 8. The light transport unit 8 is advantageous if a light source 3 is arranged separately from the beam splitter unit 6, for example at the level of the surface 10, and is to be guided to the beam splitter unit 6 with little or no loss with regard to light intensity. The light transport unit 8 can, for example, be a light guide such as a fiber optic cable, or also a mirror system or another optical system which directs the main primary beam 4.1 to the beamsplitter unit 6.

The beamsplitter unit 6 is arranged at a first distance d₁ from a first reflection region 11.1 of a first reflection unit 11. An incoming light beam is reflected at the reflection region 11.1. The first reflection region 11.1 forms a reflection plane for reflection. The first reflection unit 11 can be arranged with regard to the application for example on a surface 10 (such as on a lane of a road). The distance d₁ can also be suitably selected with regard to the application, for example depending on the vehicles passing through, but also by the direction of the measurement. The distance d₁ can for example be less if a measurement is carried out parallel to the surface 10, and can be greater when measured normal to the surface 10.

A measurement volume 2 is thereby formed between the beam splitter unit 6 and the first reflection unit 11, in which at least one gaseous and/or solid material which has been emitted by an emission source 15 is to be measured.

The main primary beam 4.1 is divided in the beam splitter unit 6 into a first partial beam 4′ and at least one secondary primary beam 13 which continue in different directions. For this purpose, a double mirror, or another suitable optical system or device, can be provided in the beam splitter unit 6, which divides the light intensity of the main primary beam 4.1 into a light intensity of the first partial beam 4″ and of the at least one secondary primary beam 13.

Advantageously, the light intensity of the main primary beam 4.1 is divided half into the light intensity of the secondary primary beam 13 and half into the first partial beam 4″.

The first partial beam 4″ is directed by the beam splitter unit 6 in the direction of the first reflection region 11.1 of the reflection unit 11. The first partial beam 4′ passes through the at least one measurement volume 2. The first partial beam 4″ is reflected from the first reflection region 11.1 and is deflected as a first return beam 14″ in the direction of at least one detector 5.

The first partial beam 4″ is preferably deflected by the beam splitter unit 6 to the first reflection region 11.1 of the reflection unit 11 such that the direction of the first return beam 14″ is opposite but parallel to the direction of the first partial beam 4″.

In this way, the first return beam 14″ again passes through the at least one measurement volume 2. The detector 5, which detects the first return beam 14″, can be arranged at any suitable location of the device 1.

In the embodiment according to FIG. 2, the first partial beam 4″ is deflected by the beam splitter unit 6 into a direction different from the direction of incidence of the main primary beam 4.1 into the beam splitter unit 6, and the secondary primary beam 13 substantially maintains the direction of incidence of the main primary beam 4.1 entering the beam splitter unit 6. In other words, the beam splitter unit 6 divides the main primary beam 4.1 into a first partial beam 4″ into a direction different from the direction of incidence of the main primary beam and a secondary primary beam 13 in a direction which substantially corresponds to the direction of incidence of the main primary beam 4.1 into the beam splitter unit 6. In particular, the first partial beam 4″ is deflected by the beam splitter unit 6 in a direction which is oriented substantially normal to the first reflection region 11.1 and/or a reflection plane of the first reflection region 11.1.

The arrangement of the first reflection unit 11 with the first reflection region 11.1 can accordingly be selected independently of the position of the detector 5. For example, the first reflection unit 11 can be mounted parallel to the plane of the surface 10, normal to the plane of the surface 10, or also in an intermediate angle to the plane of the surface 10. Advantageously, the reflection unit 11 can also be mounted in a surface 10 and can be protected from damage and contamination from emission sources 15, such as vehicles driving over the surface 10, by means of a suitable coating or installation.

It can also be provided to first deflect the first return beam 14″ from the first reflection region 11.1 back to the beam splitter unit 6. The first return beam 14″ can then be deflected by the beam splitter unit 6 in the direction of the at least one detector 5 (indicated by the primary return beam 13″ in FIG. 2). This can be done, for example, by the light transport unit 8 when the light source 3 and the detector 5 are arranged spatially together. However, it can also be provided to arrange the detector 5 spatially separate from the light source 3. In this case, the first return beam 14″ could be transmitted to the detector 5 in a suitable manner, for example via a mirror system or a separate optical waveguide. However, the detector 5 can also be arranged directly in the beam splitter unit 6.

The beam splitter unit 6 also generates the secondary primary beam 13 which is guided in the direction of at least one deflection unit 7. The direction of the secondary primary beam 13 preferably corresponds substantially to the direction with which the main primary beam 4.1 enters the beam splitter unit 6. The deflection unit 7 is at a second distance d₂ from a second reflection region 11.2 of the reflection unit 11. The distance d₂ can also be selected according to the application, for example depending on an emission source 15, but also on the direction of the measurement. The first distance d₁ and the second distance d₂ can also result from structural or natural height differences. For example, an elevation of the surface 10 or changes in the nature of the surface 10 can lead to the distances d₁, d₂ being different. Preferably, the distances d₁, d₂ are equal, but can also be different.

A measurement volume 2 forms between the deflection unit 7 and the second reflection region 11.2. This can be the same measurement volume 2 as between the beam splitter unit 6 and the first reflection region 11.1, or also a separate measurement volume.

The at least one measurement volume 2 is accordingly arranged between the beam splitter unit 6 and/or the deflection unit 7 and the associated reflection region 11.1, 11.2, and is at least partially bordered by the beam splitter unit 6 and/or the deflection unit 7 and the reflection unit 11. However, two separate measurement volumes 2 can also be provided.

The first reflection region 11.1 and the second reflection region 11.2 can be formed on a common first reflection unit 11, but can also be assigned to separate reflection units 11.

The deflection unit 7, for example a deflection mirror or another suitable optical system or device, is designed to deflect the secondary primary beam 13 as a second partial beam 4′ through the at least one measurement volume 2 in the direction of the second reflection region 11.2. The deflection unit 7 can also deflect the secondary primary beam 13 (as shown in FIG. 2).

According to the invention, the second partial beam 4′ passes through the at least one measurement volume 2. The second partial beam 4′ is reflected at the second reflection region 11.2 and is deflected as a second return beam 14′ in the direction of the at least one detector 5 and thereby passes through the measurement volume 2 a second time.

Alternatively, the second return beam 14′ can also be deflected toward the deflection unit 7, wherein the second partial beam 4′ again passes through the at least one measurement volume 2.

According to the embodiment according to FIG. 2, the secondary primary beam 13 is deflected by the deflection unit 7 in such a way that the direction of the second partial beam 4′ is oriented substantially normal to the second reflection region 11.2 and/or a reflection plane of the second reflection region 11.2. In particular, the secondary primary beam 13 is deflected by the deflection unit 7 to the second reflection region 11.2 such that the direction of the second return beam 14′ is opposite, but parallel to the direction of the second partial beam 4′.

The second return beam 14′ can be deflected in the deflection unit 7 to the detector 5 or to the beam splitter unit 6 and can be deflected like the first return beam 14″ by the beam splitter unit 6 in the direction of the at least one detector 5 (indicated by the primary return beam 13′ in FIG. 2).

The arrangement of the second reflection region 11.2 can be selected independently of the position of the detector 5 which is to detect the second return beam 14′. For example, the second reflection region 11.2 can be arranged parallel to the plane of the surface 10, normal to the plane of the surface 10, or also at an angle to the plane of the surface 10.

In a possible embodiment, multiple detectors 5 are also provided in order, for example, to detect different return beams 14′, 14″ separately.

In one possible embodiment, a detector 5 is arranged in the deflection unit 7 in order to detect the second return beam 14′ separately from the first return beam 14″. After being reflected at the second reflection region 11.2, the second return beam 14′ is then deflected toward the detector 5 in the deflection unit 7, and forwarding to another detector 5, possibly also via the beam splitter unit 6, is unnecessary. The detector 5 for the second return beam 14′ can also be arranged at any other location of the device 1, as long as the second return beam 14′ can be guided thereto. Equally, a single detector 5 can be provided for the first return beam 14″ and the second return beam 14′.

The at least one detector 5 measures at least one light property of a detected return beam 14′, 14″ which characterizes the gaseous or solid material to be measured. For example, a light intensity or a wavelength or any other measurable light property can be measured as a light property. From the measured light property, conclusions can then be drawn about the gaseous or solid material, for example about the presence of the material, a quantity or a concentration of the material.

In the device 1 according to the invention, a partial beam 4′, 4″ passes through the at least one measurement volume 2 twice due to the reflection by a reflection region 11.1, 11.2, whereby the light beam is influenced twice by the gaseous and/or solid material in the measurement volume 2, which results in an integral measurement. This can result in increased sensitivity and an improvement in the signal-to-noise ratio during measurement since larger measurement signals are possible. A greater measurement quality can accordingly be achieved.

Advantageously, the detector 5 can measure the light intensity of the detected return beam 14′, 14″ which is reduced due to the at least one gaseous or solid material and convert it into an absorption of a gaseous material, for example by means of a previously carried out reference measurement in the detector 5, which is carried out in the absence of a material in measurement volume 2. Such a reference measurement can also be done at regular intervals or as required. An attenuation of the light intensity due to a solid material, for example due to scattering, can also be detected in this way. However, an attenuation of the light intensity of the detected return beam 14′, 14″ relative to the light intensity of the partial beam 4′, 4″ belonging to the respective return beam 14′, 14″ or also relative to the main primary beam 4.1 or the secondary primary beam 13 can also be determined.

The measurement station, in particular the measurement volume 2, is stationary during the measurement with the device 1, and therefore a stationary measurement is realized with the device 1 at a specific stationary measurement station. The exhaust plume 31 can accordingly move or change relative to the measurement volume 2 during the measurement, but the device 1, specifically the individual units of the device 1, remain stationary at the measurement station. In particular, the at least one light source 3, a detector 5, a deflection unit 7, a beam splitter unit 6, and a reflection unit 11 remain stationary during the measurement. If additional units are present in the measuring unit 1, such as a multiplexer unit, a modulation unit 16, an imaging unit 29, etc., these are also stationary during the measurement.

The device 1 according to the invention is accordingly in particular not a measuring unit which is installed in a moving vehicle for exhaust gas measurement and moves along with the vehicle during the measurement.

However, the device 1 is necessarily arranged in a stationary manner only during the measurement. The device 1 or parts thereof can also be moved between two measurements. In the course of remote sensing measurements, it can for example be necessary for the device 1 to change between different measurement stations, for example in order to detect a larger part of vehicles driving in a larger area (e.g., a city). In this context, it can also be provided to mount the device 1 partially or completely on a trailer in order to be able to move the device 1 between the measurement stations. The main part of the device 1, in particular a light source 3, a detector 5, a deflection unit 7 and a beam splitter unit 6 can be preassembled on an extendable frame on the trailer. Parts of the measuring unit 1, such as the reflection unit 11 which can be attached to the measurement station are then arranged in a suitable manner at the measurement station. However, all units are again stationary during the measurement.

Advantageous embodiments of the device 1 according to the invention are described in the following.

The reflection unit 11 can, for example, be designed to be redundant, and multiple reflection units 11 can be provided per beam splitter unit 6 and/or deflection unit 7. Hence, in the event of damage or contamination of a reflection region 11.1, 11.2 of a reflection unit 11, it can be possible to direct a partial beam 4′, 4″ to another reflection unit 11, or to exchange one reflection unit 11 with another. Reflection units 11 can accordingly also be easily exchanged and serviced without having to thereby interrupt the measurement with the device 1.

One possible embodiment of protection of a reflection unit 11 is a protective film unit 24 which is shown in FIG. 9. This is for arranging a protective film 23 over a reflection unit 11 in order to protect it from contamination or damage (e.g., by scratches). The protective foil 23 is of course designed to be sufficiently transparent. A soiled protective film 23 can be replaced if necessary by a clean protective film 23. In the shown embodiment, a possible embodiment of a protective film unit 24 according to FIG. 9 consists of a first roller 20, onto which the clean protective film 23 is wound. A clean protective film 23 can be unwound from this first roller 20 and arranged above a reflection unit 11. A second roller 21 can be provided on which the soiled protective film 23 can be wound. When used as intended, a clean protective film is unwound from the first roller 20 as needed, and the soiled protective film is wound up by the second roller 21 at the same time. In this embodiment, the reflection unit 11 is arranged below the surface 10. The unwound protective film 23 is arranged above the reflection unit 11 in order to protect the reflection unit 11 from contamination or damage. For stability reasons, a mechanical protection 22 can also be provided between the reflection unit 11 and the protective film 23, which however should enable sufficient optical permeability. One of the two rollers 20, 21 can be driven in order to cause movement of the protective film 23 over the reflection unit 11 as required. An automation unit can also be provided for this purpose which controls the drive of the driven roller 20, 21. Advantageously, the rollers 20, 21 can be driven when a limit value is undershot, for example a light intensity loss of a return beam 14′, 14″ detected with a detector 5. The automation unit can then automatically control the driven roller 20, 21 in order to move the protective film 23. Accordingly, the soiled protective film 23 above the reflection unit 11 can be easily replaced with an unsoiled one as required. This can be advantageous if the reflection unit 11 is generally exposed to high contamination.

A heating device, for example an electric heater, can also be integrated into the protective film unit 24, preferably in the mechanical protection 22. The heating device can prevent the optical systems of the device 1 from being impaired by the formation of ice or puddles on the surface of the protective film 23 in wet conditions such as rain, mist and snow, for example.

It can also be provided that multiple measurement volumes 2 are formed, or each partial beam 4′, 4″ passes through a separate measurement volume 2. This can be advantageous if, for example, multiple different surfaces 10 such as a multi-lane road are measured. This can significantly increase the measurement throughput and increase the statistical safety in a measurement campaign.

FIG. 3a shows another advantageous embodiment of the device 1 according to the invention. A main primary beam 4.1 is introduced into the beam splitter unit 6, and there, as explained above, is divided into the secondary primary beam 13 and into the first partial beam 4″ of different directions. The first partial beam 4″ is deflected to the first reflection region 11.1. The secondary primary beam 13 is now forwarded, instead of to a deflection unit 7, to another second beam splitter unit 6′ which is arranged at a different, third distance d₃ (preferably equal to distances d₁, d₂) from another, third reflection region 11.3 of the reflection unit 11, wherein a further or the measurement volume 2 is again formed therebetween. This second beam splitter unit 6′ has the same function as the first beam splitter unit 6 and divides the incoming secondary primary beam 13 into another, third partial beam 4″ and another, second secondary primary beam 13″ of different directions. The third partial beam 4″ is deflected in the direction of the third reflection unit 11.3 and reflected thereon, and the reflected third return beam 14″ is forwarded to a detector 5 at which it is detected. The second secondary primary beam 13″ forwarded by the second beam splitter unit 6′ can now, as in FIG. 1, be deflected to a deflection unit 7 and deflected into the second partial beam 4′. The division of the light intensities in the beam splitter units 6, 6′ can be selected as required. It is accordingly possible to easily form additional measurement stations in the same measurement volume 2 or in different measurement volumes 2 by providing additional beam splitter units 6′.

This embodiment is particularly advantageous in order to enable multiple spatially resolved measurements of materials in at least one measurement volume 2, for example in order to measure a gaseous or solid material of an exhaust plume of an emission source 15 at multiple points. In this case, the partial beams 4′, 4″, 4″ are guided through the same measurement volume 2 in which the exhaust plume is located. Furthermore, this embodiment can be advantageous in order to detect different spatially separate measurement volumes 2 simultaneously with only one light source 3. In this case, at least two partial beams 4′, 4″, 4″ would be guided through different measurement volumes 2. It can also be advantageous to measure each return beam 14′, 14″, 14″ with a separate detector 5.

FIG. 3b shows another advantageous embodiment of the invention. The main primary beam 4.1 is introduced into the beam splitter unit 6 and divided there into two secondary primary beams 13 of different directions. The secondary primary beams 13 are oriented differently from the partial beams 4″ (not drawn). Advantageously, although the secondary primary beams 13 are oriented in different directions, the directions are in a common secondary primary beam plane which corresponds to the plane of the page in FIG. 3b. In other words, here the main primary beam 4.1 is divided into at least two secondary primary beams 13 which are oriented in different directions, but preferably run in a common secondary primary beam plane. The main primary beam 4.1 also favorably runs in this secondary primary beam plane.

In the embodiment according to FIG. 3b, the main primary beam 4.1 is divided into multiple secondary primary beams 13 which lie together with the main primary beam 4.1 in a common secondary primary beam plane.

The partial beams 4′, 4″ (e.g., normal to the drawing plane) are not shown here. Each secondary primary beam 13 can be guided either to another beam splitter unit 6′ (as in FIG. 3a) or a deflection unit 7. Different measuring arrangements can be realized with this advantageous embodiment. In a special embodiment, a measurement can accordingly be carried out parallel to the surface 10 and simultaneously normal to the surface 10, which corresponds to a multidimensional measurement in the measurement volume 2. Both, multiple measurements in the same measurement volume 2 and measurements in different measurement volumes 2, can also be realized in this way.

FIG. 4 shows another advantageous embodiment of the invention. The main primary beam 4.1 or the secondary primary beam 13, 13″ is deflected into the beam splitter unit 6, 6′ or into the deflection unit 7 (not shown) and is deflected there into a partial beam 4′, 4″, 4″ as described above. The at least one partial beam 4′, 4″, 4″ is then guided via at least one modulation unit 16. A modulation unit 16 is advantageously used for each partial beam 4′, 4″, 4″ in the device 1.

The at least one modulation unit 16 causes the at least one partial beam 4′, 4″, 4″ to be divided into individual light packets 17, if necessary also with different light intensities I. The light packets 17 have a predefined time length and are separated in time. In a simplest embodiment, such a modulation unit 16 can be a light chopper which generates defined light packets 17. Such light choppers can be, for example, rotating disks, mirrors, angle mirrors or prisms. Electro-optical modulators as a modulation unit 16, such as Mach-Zehnder interferometers, are also conceivable.

However, it is also conceivable that a return beam 14′, 14″, 14″ is modulated by the modulation unit 16, and the individual light packets 17 of the return beam 14′, 14″, 14″ are detected by a detector 5. It is also conceivable that a modulation unit 16 divides the main primary beam 4.1 or a secondary primary beam 13, 13″ into individual light packages 17′, 17″, 17″.

In any case, the division into individual light packets 17 causes the return beams 14′, 14″, 14″ detected by a detector 5 to also be divided into light packets 17. The division into individual light packages 17 is advantageously carried out in such a way that the light packets 17 arrive in the detector 5 offset in time. This allows the easy measurement of different return beams 14′, 14″, 14″ in one detector 5.

For example, in a device as in FIG. 2, both the first partial beam 4″ and the second partial beam 4′ can be divided into light packets 17″, 17″ with a modulation unit 16 each. The modulation by means of a first modulation unit 16 of the first partial beam 4″ can be performed offset in time to modulation by means of a second modulation unit 16 of the second partial beam 4′ (as indicated in FIG. 4). This means that the reflected light packets 17″ of the first partial beam 4″ arrive at a detector 5 offset in time relative to the reflected light packets 17′ of the second partial beam 4′. Accordingly, a spatially resolved measurement by a single detector 5 can be easily enabled.

FIG. 5 shows another advantageous embodiment of the invention. According to the invention, a main primary beam 4.1 is guided into the beam splitter unit 6, 6′. In one possible embodiment, the beam splitter unit 6, 6′ is designed to be pivotable and can be pivoted, for example, at an angle γ about an axis normal to a reflection region 11.1, 11.2, 11.3, or normal to a reflection plane of a reflection unit 11, or normal to a surface 10, or normal to a surface 10 on which the reflection unit 11 is arranged, and can accordingly alternately deflect the at least one secondary primary beam 13 to a plurality of stationary deflection units 7 or other beam splitter units 6′.

The beam splitter unit 6, 6′ can itself be designed to be pivotable. Advantageously, however, only a mirror in a beam splitter unit 6, 6′ can also perform the changed deflection to different deflection units 7 or additional beam splitter units 6′. In one embodiment, for example, this is a rotating shaft with mirrors at a defined distance which direct the secondary primary beams 13 to various deflection units 7 or other beam splitter units 6′. In this embodiment, the rotating shaft with mirrors also acts like a modulation unit 16 since light packets 17 are likewise generated here.

Provision can also be made for the entire device 1 to be designed pivotable. For example, beam splitter unit 6, 6′ and deflection unit 7 are fixed to the device 1, and the entire device 1 can be pivoted about an axis normal to the surface 10. The entire device 1 can then change between different lanes depending on the traffic volume, for example.

In one embodiment, a positioning optical unit 27 (indicated in FIG. 6) is additionally provided in the beam splitter unit 6, 6′ and/or in the deflection unit 7. This enables an alignment of partial beams 4′, 4″, 4″ and/or of a secondary primary beam 13, 13″ and can be used, for example, for adjusting and readjustment. A readjustment can be necessary, for example, if the beam splitter unit 6, 6′ and/or the deflection unit 7 have been misaligned by vibrations or other influences.

Such a positioning optical unit 27 can be, for example, an x-y galvanometer or also a digital mirror device. A readjustment can be necessary, for example, also in the event of contamination of a reflection region 11.1, 11.2, 11.3.

A positioning optical unit 27 can also be used in another advantageous embodiment of the invention which makes it possible to realize a plurality of passages of a light beam through the measurement volume 2, and to accordingly achieve further improvement of the measuring quality as explained with reference to FIG. 6. Opposite a reflection region 11.1, 11.2, an opposite second reflection unit 18 is arranged with a twin reflection region 30 which likewise forms a reflection plane and at which an incident light beam is reflected. The reflection region 11.1, 11.2 of the first reflection unit 11 and the twin reflection region 30 of the second reflection unit 18 are arranged facing one another. Preferably, the reflection region 11.1, 11.2 and the twin reflection region 30 are arranged parallel. In other words, the reflection planes of the reflection region 11.1, 11.2 of the reflection unit 11 and of the twin reflection region 30 of the second reflection unit 18 are arranged parallel.

The beam splitter unit 6, 6′ or the deflection unit 7 can now, for example, adjust the angle α of the partial beam 4′, 4″, 4″ via the positioning optical unit 27 to deviate from a normal to the reflection region 11.1, 11.2. The partial beam 4′, 4″, 4″ is accordingly reflected at the reflection region 11.1, 11.2 and reflected back to the opposite second reflection unit 18, where the partial beam 4′, 4″, 4″ is again reflected on the twin reflection region 30. Depending on the angle α, a plurality n of reflections can accordingly be set with which the partial beam 4′, 4″, 4″ runs back and forth between the reflection units 11, 18. This can be advantageous since the partial beam 4′, 4″, 4″ can therefore pass through a measurement volume 2 several times, and a higher sensitivity of the measurement of a gaseous or solid material in the measurement volume 2 can therefore be achieved by the integral measurement.

The at least one detector 5 can now be arranged, for example, on one of the reflection units 11, 18 and detect the partial beam 4′, 4″, 4″ after the last reflection as a return beam 14′, 14″, 14″. However, it is also possible to deflect the partial beam 4′, 4″, 4″ back to the beam splitter unit 6, 6′ or deflection unit 7 as a return beam 14′, 14″, 14′″ after a plurality n of reflections, and to pass it on there in the direction of the detector 5, whereby the detector 5 can also be arranged in the respective beam splitter unit 6, 6′ or deflection unit 7. This can be done, for example, via a deflection mirror 28. The deflection mirror 28 is preferably designed such that an incoming return beam 14′, 14″, 14″ is reflected against the direction of incidence. The direction of the beam reflected by the deflection mirror 28 accordingly coincides with the incident return beam 14′, 14″, 14″, but is oriented in the opposite direction.

The return beam 14′, 14″, 14″ can again be reflected several times between the reflection units 11, 18 (wherein the number of reflections on the way back does not have to correspond to the number of reflections on the way out), or is returned to the detector 5 via a suitable optical path after being reflected at the deflection mirror 28.

The reflection units 11, 18 with the respective reflection regions 11.1, 11.2, 30 are of course arranged and designed in a suitable manner to enable the desired reflections. Such an arrangement and design is within the ability of a person skilled in the art and can be implemented depending on the requirements of the application.

FIG. 7 shows another advantageous embodiment of the invention. According to the invention, the main primary beam 4.1 or the secondary primary beam 13, 13′ (not shown) is introduced into the beam splitter unit 6, 6′ or deflection unit 7. A multiplexer unit 19 is provided in the beam splitter unit 6, 6′ or deflection unit 7. The multiplexer unit 19 can be designed as an electrical or electro-optical circuit which divides the main primary beam 4.1 or the secondary primary beam 13, 13′ into a plurality of individual partial beams 4′, 4″, 4″ (in FIG. 7 only partially with reference signs). A partial beam 4′, 4″, 4″ is accordingly multiplied by the multiplexer unit 19. The partial beams 4′, 4″, 4″ can be guided at different points of the respective reflection region 11.1, 11.2, 11.3 associated with the beam splitter unit 6, 6′ or deflection unit 7. The partial beams 4′, 4″, 4″ can all have the same light intensity as the main primary beam 4.1 or the secondary primary beam 13, 13′. However, it is also possible for the partial beams 4′, 4″, 4″ to have different light intensities. The individual return beams 14′, 14″, 14′″ arising as described above can be detected by different detectors 5.

Each generated partial beam 4′, 4″, 4″ can be guided in the multiplexer unit 19 via a dedicated optical path 32. The optical paths 32 can have different optical path lengths, wherein each partial beam 4′, 4″, 4″ thereby receives a different optical propagation time. The individual partial beams 4′, 4″, 4″ are reflected at the reflection unit 11, and the individual return beams 14 (not shown in FIG. 7) are directed back to the beam splitter unit 6 or deflection unit 7, possibly also via the optical paths 32 of different path lengths, as described above. The at least one detector 5 can then measure the arising individual return beams 14′, 14″, 14″ more easily, because they arrive at detector 5 at successive times due to the different path lengths. This can enable a spatially resolved measurement of the gaseous or solid material in the measurement volume 2.

In another embodiment, the multiplexer unit 19 is used to generate a plurality of secondary primary beams 13 in a beam splitter unit 6, 6′ (for example in an arrangement as shown in FIG. 3b), which can be guided to additional beam splitter units 6′ and/or deflection units 7. In this embodiment, the light intensity of the plurality of secondary primary beams 13 can accordingly be the same after being split by the multiplexer unit 19.

FIG. 8 shows another advantageous embodiment of the invention for precise measurement of concentration of a gaseous or solid material in a measurement volume 2 with a device 1 according to the invention. In order to determine a concentration c of a material more precisely, knowledge is required about the actual passage length x of the light through the volume containing the gaseous or solid material (e.g., an exhaust plume) in the measurement volume 2 and about the absorption 1−(I/I₀), referred to in short as A, or transmission I/I₀, of a certain wavelength. The measurement of a material is frequency-dependent and should therefore take place at, or at least close to, the absorption maximum in order to obtain a reliable result. For example, CO₂ has characteristic vibrational oscillations at a wave number (reciprocal of the wavelength) of 1388 cm⁻¹ (asymmetrical stretching oscillation) and at 667 cm⁻¹ (bending oscillation). According to Lambert-Beer's law, the absorption A is dependent on the passage length x, the concentration c, and an absorption coefficient k (as known material parameter) using the formula

$$\\ ln \frac{I}{{I}_{0}} = k * x * c .$$

Absorption A can be determined by a detector 5. However, the passage length x is dependent on the extent of the exhaust plume 31 in the measurement volume 2 and is usually not known.

In order to detect the passage length x, according to the invention an imaging unit 29 is provided in the device 1 in order to record at least a part of the measurement volume 2 from different directions (e.g., angles ω, β). The imaging unit 29 generates images of the measurement volume 2 from different directions which are processed in an evaluation unit 26. From the obtained images from different directions, the evaluation unit 26 can then reconstruct a part of an image of an exhaust plume 31 in the measurement volume 2. The passage length x of a partial beam 4′, 4″, 4″ through the exhaust plume 31, as a sum of all passages of the light through the exhaust plume 31, can be determined from the image of the part of the exhaust plume 31. For example, the known dimensions of the measurement volume 2 can be used to calculate the dimensions of at least a part of the exhaust plume 31 in the measurement volume 2 and hence the passage length x.

For this purpose, a 2D projection of the exhaust plume 31 in the plane of the partial beam 4′, 4″, 4″ and/or of the return beam 14′, 14″, 14″ can be generated from the images, and the passage length x can accordingly be determined directly. In one embodiment, the evaluation unit 26 creates a spatial reconstruction of the exhaust plume. This reconstruction can also be dependent, for example, on a running variable, such as the time. Accordingly, for example, a time-dependent expansion of an exhaust plume 31 can be determined.

In another embodiment, the evaluation unit 26 receives data about the outside temperature and air humidity. Depending on the outside temperature and air humidity, there can be differences in the evaluation and reconstruction of an exhaust plume. For example, temperature differences in the summer between the surroundings and the exhaust plume are less pronounced than in winter. This can cause the passage length x to have seasonal differences. In order to avoid this source of error, a correction factor can be provided for calculating the reconstruction depending on the outside temperature and the air humidity. The evaluation unit 26 can accordingly carry out a reliable calculation of the passage length x independent of the conditions.

The evaluation unit 26, usually a computer with corresponding evaluation software, can also receive data for absorption A from at least one detector 5, and the passage length x, which was reconstructed from the part of an image of the exhaust plume 31, can be used to calculate the concentration c of a material according to Lambert-Beer's law. In an advantageous embodiment, multiple data of the absorption A can be used in order to calculate a spatial distribution of the concentration c in a measurement volume 2.

The imaging unit 29 can be designed in the form of multiple cameras 25 (as shown in FIG. 8). An embodiment of the imaging unit 29 with one or more lidar units, one or more radar units or combinations of such units or with cameras is also conceivable. In addition, it is of course also possible for there to be additional embodiments of an imaging unit 29.

In one embodiment with cameras 25, these are installed at different locations in order to record a measurement volume 2 from different directions w, B. The cameras 25 can, for example, be arranged on a beam splitter unit 6, 6′ and/or deflection unit 7. The cameras 25 can also be installed on a separate device, or can use existing infrastructure in the region of the device 1 such as bridges, houses, street lights or the like. When there is a plurality of beam splitter units 6, 6′ and/or deflection units 7, the cameras 25 can also be arranged such that they can simultaneously detect multiple measurement volumes 2, for example. The number of cameras 25 can accordingly be kept low.

The cameras 25 can record images of the measurement volume 2 and accordingly also record an exhaust plume 31 present in the measurement volume 2. However, it is also possible for the cameras 25 to additionally record metadata of a vehicle, such as size, type, or also the license plate. When cameras 25 are used as the imaging unit 29, image processing software such as ImageJ can be used, for example, in order to reconstruct the exhaust plume 31 or a part thereof.

The cameras 25 can, for example, be infrared cameras which record heat images of the present exhaust plume in the measurement volume 2. The heat distribution in the exhaust plume can accordingly also be detected, which can have an influence on the materials or the absorption coefficients k. Due to the temperature differences, convection and diffusion phenomena can occur which cause a distribution of materials over time. Individual concentrations c of materials can also be dependent on the temperature, since some reactions proceed only at a higher temperature. It can also be possible for different exhaust plumes from sequential or adjacent emission sources 15 such as vehicles to be mixed. The measurement can then be accordingly adapted, for example using the above-described positioning optical unit 27, or a positioning or alignment of a camera 25.

However, the cameras 25 can also operate in the ultraviolet (UV) or visible (VIS) range, for example, or also in both ranges (UV/VIS cameras). UV or VIS is higher energetic radiation than IR and excites electron transition in molecules and can be more advantageous for measurement.

In one possible embodiment, the cameras 25 are designed as multi- and hyperspectral cameras. In this case, instead of classic simple recording in a simple spectral range, a high number of spectral bands are used. This can be advantageous in order to detect a significantly higher color quality and color differences, since each pixel already contains a complete color spectrum. Such a camera 25 can function, for example, using the snapshot mosaic technique.

A lidar unit is based on a laser, for example a YAG laser with 1064 nm or 532 nm wavelength, or similar designs that a person skilled in the art deems suitable. IR lasers can also be used, although sufficient shielding can be necessary in order to avoid eye damage. A lidar unit in the UV or NIR (near-infrared) range can, for example, be used to also measure gaseous or solid materials directly. As is known, lidar can also detect for example carbon dioxide (CO₂), sulfur dioxide (SO₂) and methane (CH₄) from atmospheric measurements. This can be used to carry out, for example, rough estimations of materials or also to obtain redundant measurements for the measurement according to the invention.

The at least one lidar unit can move in at least one axis and record images of the surroundings and the present exhaust plumes 31. The at least one lidar unit can be used to image different exhaust plumes 31 in a measurement volume 2, or also different exhaust plumes 31 in different measurement volumes 2. For this purpose, the lidar unit scans the surroundings and, depending on the reflection time of the emitted laser pulse, images of the surroundings can be generated.

A combination of lidar units and cameras 25 is also conceivable as an imaging unit 29. Accordingly, for example, gaseous materials can be measured via a lidar unit, while solid materials in the exhaust plume 31 are detected via the device 1 according to the invention. This allows a representative concentration measurement of several critical materials in the exhaust plume 31.

It is also possible for a camera image to be taken in the region of the device 1 in addition to the measurements described above. In this way, subject to data protection requirements, license plates of vehicles can be recorded. Vehicle owners can accordingly be notified if a vehicle has materials in the form of exhaust emissions outside standard reference values.

1. A device for measuring at least one gaseous or solid material in at least one measurement volume at a stationary measurement station, wherein a light source and at least one detector are provided, and at least one main primary beam is emittable from the light source to at least one beam splitter unit,
wherein the at least one beam splitter unit is arranged at a first distance from a first reflection region of a reflection unit, and the beam splitter unit splits the main primary beam into at least one first partial beam oriented through the measurement volume toward the first reflection region and at least one secondary primary beam oriented in a different direction than the main primary beam,
wherein at least one deflecting unit is arranged at a second distance from a second reflection region, and the at least one secondary primary beam can be directed to the deflecting unit by means of the beam splitter unit and the at least one secondary primary beam can be directed, as a second partial beam, through the at least one measurement volume toward the second reflection region by means of the deflecting unit,
wherein the at least one measurement volume is arranged between the beam splitter unit or the deflection unit and the associated reflection regions, and is at least partially delimited by the beam splitter unit or the deflection unit and the associated reflection regions,
wherein the first reflection region is provided in order to direct the first partial beam as a first return beam through the at least one measurement volume to the at least one detector, wherein the second reflection region is provided in order to direct the second partial beam as a second return beam through the at least one measuring volume to the at least one detector, and wherein the at least one detector is provided to measure a light property of each return beam characterizing the at least one gaseous or solid material.

2. The device according to claim 1, wherein the reflection regions are configured to direct the respective return beam to one detector each.

3. The device according to claim 1, wherein the first return beam is deflected back from the first reflection region via the beam splitter unit to the at least one detector.

4. The device according to claim 1, wherein the second return beam is deflected back from the second reflection region via the deflection unit, or via the deflection unit and the beam splitter unit, to the at least one detector.

5. The device according to claim 1, wherein the at least one detector is arranged in the deflection unit or the beam splitter unit, or a detector is arranged in the deflection unit or the beam splitter unit in each case.

6. The device according to claim 1, wherein the at least one detector is installed with the light source in a housing.

7. The device according to claim 1, wherein at least one second beam splitter unit at a third distance from a third reflection region is provided, wherein the second beam splitter unit is arranged between the first beam splitter unit and the deflection unit and receives the secondary primary beam from the first beam splitter unit, and wherein the second beam splitter unit divides the received secondary primary beam into a third partial beam oriented in the direction of the third reflection region, and a second secondary primary beam oriented in the direction of the deflection unit.

8. The device according to claim 1, wherein the beam splitter unit divides the obtained main primary beam or secondary primary beam into a first partial beam or third partial beam and into multiple secondary primary beams oriented in different directions, wherein the beam splitter unit directs each secondary primary beam to another beam splitter unit or a deflection unit.

9. The device according to claim 1, wherein at least one beam splitter unit is arranged so as to be pivotable about an axis in order to selectively guide the generated secondary primary beam to a plurality of deflection units or additional beam splitter units, wherein the axis is designed to extend normally to a reflection region or to a surface on which the reflection unit is arranged.

10. The device according to claim 1, wherein a positioning optical unit is provided in at least one beam splitter unit or the deflection unit, and the positioning optical unit directs the first partial beam oriented in the direction of the respective reflection region in an angle deviating from a normal to the respective reflection region.

11. The device according to claim 10, wherein in at least one beam splitter unit or deflection unit an opposite second reflection unit with an opposite twin reflection region facing the respective reflection region is arranged at a distance from the respective reflection region, wherein the respective reflection region reflects the partial beam to the opposite twin reflection region, and the opposite twin reflection region reflects the partial beam back to the respective reflection region, wherein the at least one detector is configured to detect the partial beam after a plurality of such reflections as a return beam, or a deflection mirror is provided which deflects the partial beam after a plurality of such reflections as a return beam, and the at least one detector detects the return beam after a number of reflections between the reflection unit and the opposite reflection unit.

12. The device according to claim 10, wherein the angle is adjustable by the positioning optical unit.

13. The device according to, wherein at least one modulation unit is provided in the device in order to divide a beam selected from the group consisting of the partial beam, a return beam, the main primary beam, and a secondary primary beam into individual light packets.

14. The device according to claim 13, wherein at least two light packets are offset in time.

15. The device according to claim 1, wherein a multiplexer unit is provided in at least one beam splitter unit or deflection unit, and divides the obtained main primary beam or secondary primary beam into a plurality of partial beams, and the multiplexer unit directs the plurality of partial beams to different locations of the respective reflection region which reflects the plurality of sub-beams as a plurality of return beams and transmits the plurality of return beams to the multiplexer unit, and that the multiplexer unit transmits the plurality of reflected return beams to at least one detector.

16. The device according to claim 15, wherein a dedicated detector is provided for at least two of the plurality of return beams.

17. The device according to claim 15, wherein at least two of the plurality of partial beams in the multiplexer unit are each guided via a dedicated optical path, wherein the optical paths have different optical path lengths, and in that a common detector is provided for the at least two resulting return beams.

18. The device according to claim 1, wherein at least one imaging unit is provided in the device in order to record at least a part of the exhaust plume from different directions in the presence of an exhaust plume in the measuring volume, wherein an evaluation unit is provided in order to reconstruct the images recorded by the image unit into an image of the at least one part of the exhaust plume and to determine from the image of the at least one part of the exhaust plume a passage length of the partial beam or of the return beam through the exhaust plume in the measuring volume, wherein the at least one detector which detects the return beam detects a decrease in intensity of the return beam due to the at least one gaseous or solid material, and the evaluation unit is provided to determine a concentration of the at least one gaseous or solid material in the measuring volume from the decrease in intensity and the determined passage length.

19. The device according to claim 1, wherein a protective film is arranged replaceably above a reflection region.

20. A method for measuring at least one gaseous or solid material in at least one measurement volume at a stationary measurement station, which measurement volume is formed between a beam splitter unit and a first reflection region or a deflection unit and a second reflection region,
wherein a main primary beam is emitted from a light source and the main primary beam is guided to the at least one beam splitter unit in which the main primary beam is split into at least one first partial beam oriented in the direction of the first reflection region and through the at least one measuring volume and a secondary primary beam,
wherein the secondary primary beam is deflected in the deflection unit into a second partial beam oriented in the direction of the second reflection region and through the at least one measurement volume,
wherein the first partial beam is reflected at the first reflection region and is deflected back as a first return beam through the at least one measurement volume to the at least one detector,
wherein the second partial beam is reflected at the second reflection region and is deflected back as a second return beam through the at least one measurement volume to the at least one detector,
and wherein a light property of each return beam that characterizes the at least one gaseous or solid substance is measured with the at least one detector.
